(19) United States
(12) Patent Application Publication
(10) Pub. No.: US 2010/0010637 A1
(43) Pub. Date: Jan. 14, 2010

(54) TRAPEZO-METACARPAL IMPLANT

(76) Inventor: Jean-Pierre Pequignot, Nice (FR)

Correspondence Address: LUCAS & MERCANTI, LLP, 475 PARK AVENUE SOUTH, 15TH FLOOR, NEW YORK, NY, 10016, US

(21) Appl. No.: 12/526,232

(22) PCT Filed: Feb. 1, 2008
(86) PCT No.: PCT/EP08/51255

§ 371 Date: Aug. 6, 2009

(30) Foreign Application Priority Data
Feb. 7, 2007 (FR) 07/00850

(51) Int. Cl. A61F 2/42

(52) U.S. Cl. 623/21.15

(57) ABSTRACT

This present invention concerns a trapezian or trapezo-metacarpal implant (1) that includes a rod (2) and a head (3) that are respectively intended to be inserted into the metacarpus (4) of the thumb, and to be anchored in a space that is created in particular by removing at least a portion of the trapezius (5). According to the invention, the rod (2) and the head (3) of the implant are independent of each other, and are designed to fit together with each other at least at one end (2a) of the rod, through the use of complementary connecting means (6, 34) allowing movement of the head with at least one degree of freedom in rotation, in relation to the rod and the said head (3), including rotation means (32) in the said space obtained by removing at least a portion of the trapezius (5).

### FIELD OF THE INVENTION

This present invention concerns a trapezo-metacarpal implant. This implant includes a rod that is intended to be inserted into the metacarpus of the thumb, and which is terminated by a head that is intended to take up a position in a space that is created in particular by removing a part of the trapezium.

The implant of the invention can also be employed as a trapezian implant in the event of total ablation of the trapezium, and of replacing the latter by a suitable prosthetic part.

The implant according to the invention can be used to treat phenomena of arthrosis at the base of the thumb, also called root arthrosis, which is a essentially trapezo-metacarpal affection.

### THE PRIOR ART

The very specific mobility and stability of the trapezo-metacarpal joint, that is the control joint of the thumb, are associated above all with the concordance of the articular surfaces and with the integrity of the ligaments. In the event of arthrosis, these two requirements are no longer respected, whether because of wear with partial dislocation of the first metacarpal, or because of a more centred affliction, described as fusion.

Several types of trapezian and/or trapezo-metacarpal implants are known at this time, and are or were used in the surgical area in order to solve the problems associated with arthrosis of this joint.

Amongst the best known of these is the so-called Swanson trapezian implant, composed of a rod that is terminated by a head, the whole being made from silicone. In this implant, the head is of a size and shape that is designed to replace the entirety of the trapezium. This implant allows the length of the column of the thumb to be maintained by substitution of a space filling the hole that is left following the execution of a trapezectomy.

However this implant has two main inconveniences. Firstly, the material from which it is made is unsuitable. In fact, the silicone causes an allergy in some patients known by the name of siliconite. Moreover, because of its excessively low hardness, the silicone wears on contact with the adjoining bones, causing damage to the implant.

In addition, the Swanson implant is not stable and, in particular, has a tendency to dislocate or partially dislocate.

It has also been attempted, with no conclusive result, to restore the anatomical surfaces of the trapezo-metacarpal with one or two osteointegrated or cemented prosthetic elements. However, this implies an integrity or a perfect reconstruction of the ligamentary apparatus that can be verified only after operation on, and postoperative recovery of the treated patients (see Péquignot J P, Giordano Ph, Boatier C, Allieu Y. Traumatic dislocation of the Trapezo-metacarpal: Ann. Chir. Main 1988; 7: No. 1:14-24).

Coupled prostheses of the “swivel” type, such as the Maïa®, Arpe® and Electra® prostheses, offer amplitudes and a kinematic that are very close to healthy subjects. However there remains the problem of the dislocations and especially the risk of unsticking of one of the components, generally through trapezian failure. In fact, the concentration of the stresses at the level of the spongy bone through a cylindrical or conical acetabulum can only lead to embedding or bone rupture in the more-or-less long term. On the other hand, the metacarpal part remains better tolerated or even too well osteointegrated despite some structural inconsistencies.

One can also mention the anatomical prostheses that aim to reproduce the anatomical surfaces and the actual mobility of the trapezo-metacarpal joint as closely as possible. Document U.S. Pat. No. 5,405,400 (Linscheid) describes an example of an anatomical prosthesis formed from a trapezian part and a metacarpal part, each with a head in the shape of a saddle and mating with each other.

However, these prostheses also suffer from significant problems of dislocation and/or unsticking, which frequently call for a fresh surgical operation (see Phaltankar P M, Magnussen P A: Hemiarthroplasty for Trapeziometacarpal arthritis—a useful alternative? J Hand Surg. 2003; 28B, 80-85).

### OBJECTIVES AND SUMMARY OF THE INVENTION

Recent in vivo studies have allowed us to determine the articular model of the trapezo-metacarpal corresponding to a universal joint, the two axes of rotation of which are intersecting but not perpendicular, and equidistant by only a few millimetres.

In fact, the trapezo-metacarpal joint allows complex movements such as the circumduction of the first metacarpal on the trapezius, which include sliding and rotation in the three planes of the space of the head of the first metacarpus against the trapezium. However, the trapezo-metacarpal joint also allows rotation of the first metacarpus about its own longitudinal axis, in particular during pollici-digital prehension (pinching) movements, in order that the pulp or flesh of the thumb is always in pronation against the pulp of the other fingers of the hand.

At the present time, no trapezo-metacarpal joint prosthesis allow one to fully reproduce all of the natural movements of this joint, without giving rise to problems of dislocation, wear, or unsticking of the joint prosthesis.

It is for this reason that this present invention aims to provide a trapezo-metacarpal implant that is mobile and stable, and that requires a minimal osseous resection.

This present invention further aims to provide a trapezo-metacarpal implant that allows a mobility and stability of this joint that is as close as possible to the physiology, and in particular to provide an implant that is not only suitable to reproduce a non-anatomical articular model after implantation, but also one that is kinematically equivalent to the trapezo-metacarpal joint.

These objectives are attained, according to the invention, by a trapezo-metacarpal implant that includes a rod and a head that are respectively intended to be inserted into the metacarpus of the thumb and to be anchored in a space that is created in particular by removing at least part of the trapezium. This implant is characterised by the fact that the rod and the head are independent of each other, and are designed to fit together with each other at least at one end of the said rod, through the use of complementary connecting means, the said complementary connecting means allowing movements of the said head with at least one degree of freedom in rotation in relation to the said rod and the said head comprising rotation means in the said space created by removing at least part of the trapezium.

According to the invention, the ability to pivot the head of the implant on two intersecting axes or centres of rotations and not perpendicular affords greater stability to the trapezo-metacarpal implant, as well as greater mobility. A first axis or centre of rotation is located in the trapezium, the other in the base of the first metacarpal itself, and both are equidistant by a few millimetres. The mobility of the implant is thus doubled, which increases the amplitude of the movements but which in particular avoids a cam effect that is encountered in the prostheses with anatomical surfaces.

Because of the intrinsic mobility of the head of the implant in relation to its rod, the stability of this implant is better than a rigid mono-block implant, and there exists an automatic recentring of the parts during pollici-digital prehension movements, which is the major element for conversion of the partial dislocation forces into axial compression forces by automatic varisation-flexion. This automatic recentring, especially in retroposition, is a major element in the stability of the implant.

The mobility of the head in relation to the rod moreover allow to insert the rod into the metacarpus in a straight manner, that is along the longitudinal axis of the latter. The rod can therefore be placed exclusively in the soft central portion of the bone, which facilitates the work of the surgeon, who does not have to excavate in hard peripheral portion. Such positioning of the rod also avoids embrittlement of the bone to an excessive extent.

As indicated previously, the implant of the invention can also be employed as a trapezian implant in the event of trapezectomy. An articular part to replace the trapezius, frequently described as a “spacer”, is then used in place of the bone, against which the head of the implant of the invention bears and pivots, with the head itself articulating with the rod via the said complementary connecting means. This replacement part must then be stabilised by peripheral ligament reconstruction and, as a result of its shapes, it retains a certain mobility against the surrounding bones, in particular the trapezoid and scaphoid.

In an advantageous configuration of the implant of the invention, the said complementary connecting means of the rod and of the head respectively include a female portion such as a receiving orifice formed at a said end of the said rod, and a male portion such as a stud formed on the said head.

These connecting means have the advantage of being of a shape that is very easy to create while still perfectly performing the functions incumbent upon them, in particular the creation of an axis of rotation of the head in relation to the rod, and perfect transmission of the forces between the trapezius and the first metacarpal.

In a preferred manner, the said receiving orifice of the head presents a cross section to the rod that is substantially ovoid, and a longitudinal section to the rod that is substantially tapered, which is especially designed to receive a male portion of the said head of complementary shape.

The ovoid and tapered shape of the orifice for insertion of the head onto the rod confers a second degree of mobility to the head in relation to the rod, about an anteroposterior or dorso-palmar axis of the prosthetic joint. This mobility further improves the positioning and the recentring of the parts of the implant for better transmission of the forces and better compliance with the exact physiological mobility of the joint.

According to another preferred characteristic of the implant of the invention, the said rod includes a peripheral pad that is intended to rest on the proximal end of the metacarpus in which the said rod is inserted. The said pad is advantageously shaped to the periphery of the receiving orifice of the said head on the said rod.

By virtue of this peripheral pad and its anatomical shape, the rod of the implant can be inserted, after a minimal cut-back of the first metacarpal and, stabilised by a so-called “press-fit” effect, that is through pressure that blocks the rod on the resection of the metacarpus. It also presses its rounded pad against the cut-back executed at the base of the first metacarpal, thus avoiding any excessive embedding.

The head of the implant of the invention also presents a particular and advantageous shape. It includes a base that is substantially flat, preferably circular, surmounted by a substantially spherical cap with a large radius of curvature. From the flat base, on the side opposite to that of the cap, there lies a cylindrical or preferably tapered cylindrical stud, forming a said attachment means from the head to the said rod.

By a large radius of curvature of the cap is here meant a radius of curvature corresponding substantially to the radius of curvature of the trapezo-metacarpal joint. In practise, this radius of curvature is preferably between 8 and 12 millimetres. The surgeon therefore does not need to execute too great a cut at the proximal end of the metacarpus, or to excavate deeply into the trapezius, for which the removal of a small cap of large radius of curvature can suffice.

In addition, the spherical cap of the head gives it a biconvex profile, allowing multiple movements against the trapezius (tilt, sliding and rotation).

The insertion stud of the head is preferably centred in relation to the base of the head and lies on an axis that is substantially perpendicular to it. The diameter of this stud is preferably between 3 and 5.5 millimetres and its length is, also preferably, between 5 and 7.5 millimetres.

For its part, the base of the said head has a diameter that can be between about 10 and 20 millimetres. However a diameter of between 12 and 16 millimetres is preferred. The said base covers and presses against the peripheral pad of the rod when the head is inserted by its stud into the orifice formed in the rod, which facilitates the freedom of movement of the head in relation to the rod of the implant.

The material from which the implant of the invention is made must very obviously be biocompatible. In this regard, Pyrocarbon (PyC) is the favoured material from which to create this implant.

This material, which is osteo-inducing but not osteo-integrable, in fact has a very low friction coefficient that allows it to slide without adhesion to the bone with which it is in contact, and therefore without engendering any wear in the long term. Thus, even several years after implantation, no fragmentation of the surface of the implant or of the bone is observed, thus preserving perfect integrity and comfort of the prosthetic joint for the patient.

Moreover, unlike the silicone, which is too soft, and titanium, which is too hard, Pyrocarbon has an elasticity modulus, also known as Young's Modulus, that is close to that of the bone, which guarantees excellent transmission and distribution of the reciprocal forces exerted on the implant and on the adjoining bones, thus reducing the risk of pain for the patient.

Other particular characteristics of this present invention will emerge on reading the detailed description that follows of one particular embodiment of this implant, which is provided with reference to the appended drawings.

### BRIEF DESCRIPTION OF THE DRAWINGS

In the appended drawings:

FIG. 1 represents the trapezo-metacarpal implant of the invention positioned in the hand of a patient, represented partially as seen from the dorsal side, with the trapezo-metacarpal joint in the rest position seen from the front;

FIG. 2 represents the implant of the invention seen in profile;

FIG. 3 represents a frontal view of the proximal end 2a of the rod of the implant in cross section on the axis III-III in FIG. 2;

FIG. 4 is a view similar to that of FIG. 2, and in which is represented the mobility range of the head of the implant about an anteroposterior axis in relation to the rod.

### DETAILED DESCRIPTION OF ONE EMBODIMENT OF THE INVENTION

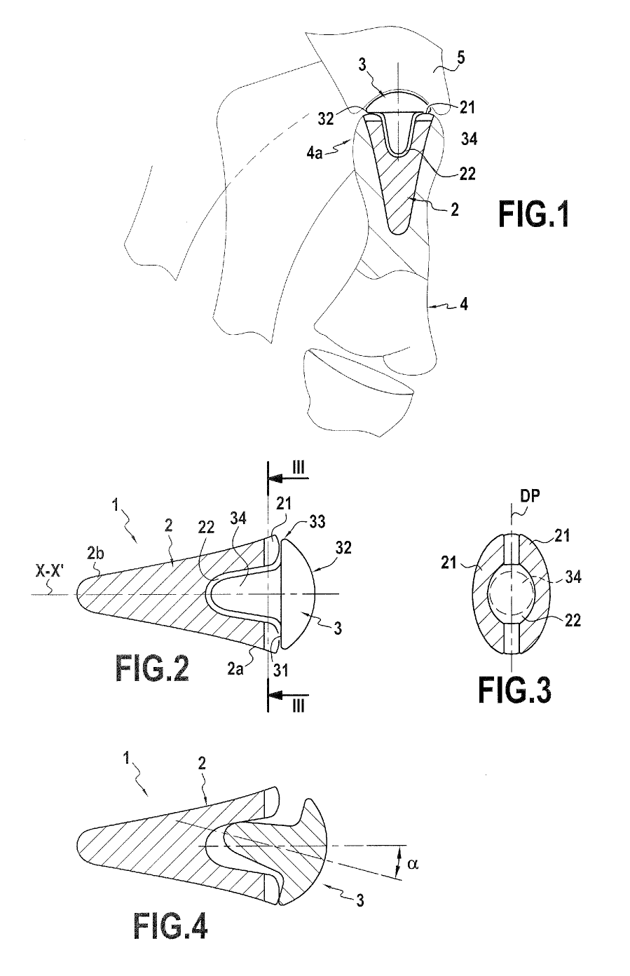

As shown in FIGS. 1 and 2, the implant 1 of the invention is a trapezo-metacarpal implant, that comes in the general shape of a stud. It includes a rod 2 which is terminated, at a so-called proximal end 2a, by a head 3, and of which the opposite so-called distal end 2b is composed of a point.

The rod 2 is intended to be inserted into the soft medullar portion of the first metacarpus 4 of the thumb, and the head 3 is intended to be anchored in a space that is created firstly by making a straight cut or resection in the proximal end 2a of the metacarpus, and secondly by removing a portion of the trapezius 5 facing the proximal portion of the metacarpus.

In the context of a trapezian implant (not shown in the figures), the entirety of the trapezius 5 is removed and replaced by a prosthetic part, also called a “spacer”, forming an acetabulum designed to receive the head of the implant, and that is secured by ligament reconstruction.

The rod 2 is matched to the profiles of the medullar cavity of the first metacarpal 4. It has a substantially triangular longitudinal section that is relatively flat in profile in its upper portion and concave in its lower (palmar) portion.

The said rod 2 is terminated at the periphery of its proximal end 2a by a pad 21, which ensures a better fit with the cut-back in the base of the metacarpal during embedding of the rod 2 in the said metacarpal 4.

As can be seen particularly in FIG. 3, the rod 2 is also excavated at its centre with an oval-shaped, large-axis DP anteroposterior dorso-plantar orifice 22 directed along the largest dimension of the said orifice 22, which is intended to receive the head 3 of the implant. This orifice 22 has a longitudinal section that is substantially tapered (FIGS. 2 and 4), slightly larger at the opening of the orifice 22 than at its bottom, so as to be able to easily receive the head 3 of the implant, as will be described below, and to allow slight tilting movements of the head 3 in the said orifice 22 about the anteroposterior dorso-plantar axis DP of the latter. As identified in FIG. 4, the amplitude of these anteroposterior tilting movements of the head are of the order of an angle α of 30° to 45° in relation to the longitudinal axis XX′ of the rod 2 of the implant.

The said head 3 includes a flat and circular base 31 that comes into contact with the top surface of the pad 21 formed at the proximal end 2a of the rod 2. Opposite to this base 31, the said head 3 includes a distal top cap 32 acting as a contact and friction surface with the remaining portion of the trapezius 5. A rounded annular edge 33 effects the connection of the said cap 32 to the base 31 of the head 3.

Only the cap 32 of the head is liable to be in contact with the remaining portion of the trapezius 5. This cap 32 has a hemispherical profile that allows sliding, rotation and pivoting movements of the head in the recess created in the trapezius 5 to receive the said head.

The head 3 is inserted into the orifice 22 of the rod 2 and is centred in the latter by a cylindrical stud 34 that is centred perpendicularly to the base 31 of the head 3. This cylindrical stud has a length and a diameter that are slightly less than those of the orifice 22, in order to allow rotary movements of the head 3 in the orifice 22 substantially about the longitudinal axis XX′ of the rod 2, which constitutes an essential characteristic of the implant of the invention.

The thickness E and the diameter D of the head 3, the radius of curvature R of the cap 32 of the head, and the length l and the diameter d of the stud 34 allowing the insertion of the head 3 onto the rod 2, depend on the size of the implant 1. In fact, at least four different sizes of implant are provided, in order to be able to adapt the said implant to the morphology and to the bone structure of the treated patients.

The diameter D of the head 3 and the radius of curvature R of the cap 32 are chosen to be sufficiently large to conform as well as possible to the anatomical curvature of the trapezo-metacarpal joint.

In practice, the diameter D of the head 3 is between 10 and 20 millimetres, and preferably between 12 and 16 millimetres. The radius of curvature R of the cap 32 is advantageously between 6 and 12 millimetres, and preferably between 8 and 11 millimetres.

For its part, the thickness E of the head 3, measured between its base 31 and the highest point of the cap 32, is preferably between 4 and 8 millimetres.

The dimensional characteristics of the stud 34 are also variable according to the size of implant concerned. The length l of the said stud 34 is thus between 5.5 and 7.5 millimetres, for a diameter d that is preferably between 3 and 5.5 millimetres.

The rod 2 and the head 3 of the implant 1 thus form two associated prosthetic parts that are independent and mobile in relation to each other, in particular in rotation around the longitudinal axis XX′ of the rod 2, by virtue of the stud 34 of the head 3 of the implant inserted into the orifice 22 created at the proximal end 2a of the rod 2 of the implant.

Moreover, by virtue of the spherical profile of the cap 32 of the head 3, the implant is also mobile to rotate, to slide and to pivot against the trapezium, which ensures a mobility and stability of the prosthetic joint that is as close as possible to the physiology, in particular allowing the necessary movements of rotary circumduction of the thumb.

The mobility of the implant 1 is doubled by virtue of the two rotation surfaces created by the stud 34 and the cap 32, which increases the amplitude of the movements while still allowing the partial dislocation forces to be recentred into compression forces by automatic varisation-flexion of the implant during pollici-digital prehension. This automatic recentring, especially in retroposition, is a major element of the stability of the implant.

In order to guarantee perfect functional integrity of the implant over time, and in order to facilitate postoperative recovery and to avoid any later complications for the treated patients, the implant 1 according to the invention is advantageously made entirely from polished Pyrocarbon (PyC).

In fact this material has many advantages in the context of implanting applications, in comparison with other materials for medical applications. Firstly, the Pyrocarbon is a material that is perfectly bio-compatible, and that also has excellent mechanical characteristics that are very close to the natural characteristics of the bone.

In particular, Pyrocarbon has an elasticity modulus (Young's Modulus) that is similar to that of the bone, between 20 and 25 GPa, which guarantees excellent transmission of the forces between bone and implant, free of any pain for the patient. It also has an extremely low friction coefficient and a high resistance to fatigue, which guarantee absence of wear and breaks in the long term, and therefore absence of inflammation for the treated patients.

Pyrocarbon is therefore particularly well suited to creation of the implant of the invention, which is intended for replacing the particularly mobile and hard-worked joint that is the trapezo-metacarpal.

The implant 1 according to the invention is positioned as follows:

After incision and opening of the tissues covering the joint to be replaced, a straight cut is made in the proximal end 4a of the metacarpus 4 and a portion of the trapezius 5 located facing this proximal end is removed, by milling for example, in order to create a space designed to receive the head 3 of the implant 1.

Next, a straight channel is shaped in the medullar portion of the metacarpus 4, along the longitudinal axis of the latter. The rod 2 of the implant is then forced into the channel after preparation by means of calibrated rasps, until the pad at the proximal end 2a of the rod 2 fits into the section in the metacarpus 4 and is trapped against the latter, without cement, by a “press-fit” effect.

Then the head 3 of the implant is positioned by inserting the stud 34 of the latter into the orifice 6 of the rod 2 of the implant, and by positioning the cap 32 of the said head against the removed portion of trapezius 5. This spherical cap 32 has a large radius of curvature that allows a broad contact surface to be created with the trapezius bone, and thus eliminates any risk of embedding or wear. The bone/prosthesis congruence is obtained by minimal centred milling of the trapezius to create a new joint. The latter certainly only allows small movements, but by virtue of a large contact area, the pressure stresses are better distributed.

Next, the implant is stabilised by a ligament reconstruction of the peripheral type, allowing the resting position of this joint to be found and then, to finish, comes the capsular closure of the tissues and suturing of the wound.

As mentioned previously, it is also possible to use the implant of the invention after a complete trapezectomy, replacing the trapezius with a prosthetic replacement part that is stabilised by peripheral ligament reconstruction which, by virtue of its shapes, will retain a certain mobility against the surrounding bones (trapezoid and scaphoid) and against which the head of the implant rests and articulates.

By the dissociation of the rod and the head of the implant, the implant of the invention thus procures a double rotational mobility of the prosthetic or replaced trapezo-metacarpal joint, in conformity with the natural joint, which ensures a mobility and a stability of this prosthetic joint that is as close as possible to the physiology, and with better postoperative recovery.

1.-11. (canceled)

12. A trapezian or trapezo-metacarpal implant that includes a rod and a head, which are respectively intended to be inserted into the first metacarpal of the thumb and to be anchored in a space that is created by removing a portion of the trapezium, wherein the said rod and the said head are independent of each other and are designed to fit together with each other at least at one end of the said rod, through the use of complementary connecting means, the said complementary connecting means allowing movements of the said head with at least one degree of freedom in rotation in relation to the said rod, and the said head including rotation means in the said space created by removing one part of the trapezius.

13. An implant according to claim 12, wherein the said complementary connecting means include a female portion formed at a said end of the said rod, and a male portion formed on the said head.

14. An implant according to claim 12, wherein the said connecting means of the rod include an orifice to receive the said head, with a cross section that is substantially ovoid, and with a longitudinal section that is substantially tapered, designed to receive a male portion of the said head of complementary shape.

15. An implant according to claim 12, wherein the said rod includes a peripheral pad intended to rest on the proximal end of the metacarpal in which the said rod is inserted.

16. An implant according to claim 15, wherein the said pad is shaped to the periphery of the receiving orifice of the said head on the said rod.

17. An implant according to claim 12, wherein the said head includes a base that is substantially flat and preferably circular, surmounted by a cap that is substantially spherical, and a stud that is preferably cylindrical, extending from the said base and forming a said attachment means from the head to the said rod.

18. An implant according to claim 17, wherein the said stud is centred in relation to the said base of the head, and lies on an axis that is substantially perpendicular to it.

19. An implant according to claim 17, wherein the said stud has a diameter of between 3 and 5.5 millimetres, and a length of between 5.5 and 7.5 millimetres.

20. An implant according to claim 17, wherein the said cap has a radius of curvature (R) of between 8 and 11 millimetres.

21. An implant according to claim 17, wherein the base of the said head has a diameter (D) of between 10 and 20 millimetres, and preferably between 12 and 16 millimetres.

22. An implant according to claim 12, wherein the said rod and the said head are made from Pyrocarbon.
